(19) United States
(12) Patent Application Publication
(10) Pub. No.: US 2013/0330798 A1
(43) Pub. Date: Dec. 12, 2013

(54) METHOD FOR FACILITATING GENERATION OF HYDROGEN BY PHOTOSYNTHETIC BACTERIA WITH ORGANIC WASTEWATER

(71) Applicants: Yinguang Chen, Shanghai (CN); Yuxiao Zhao, Shanghai (CN)

(72) Inventors: Yinguang Chen, Shanghai (CN); Yuxiao Zhao, Shanghai (CN)

(21) Appl. No.: 13/982,329

(22) PCT Filed: Jul. 1, 2011
(86) PCT No.: PCT/CN2011/076764

§ 371 Date: Aug. 30, 2013

(30) Foreign Application Priority Data
Jan. 27, 2011 (CN) 201110029589.4

(51) Int. Cl. C12P 3/00

(52) U.S. Cl. CPC C12P 3/00
USPC 435/168

(57) ABSTRACT

Provided is a method for facilitating generation of hydrogen by photosynthetic bacteria with organic wastewater by nano titania, comprising the following specific step: (1) adding organic wastewater to a photosynthetic reactor; (2) adding hydrogen generating photosynthetic bacteria to the photosynthetic reactor; (3) adding nano titania to the photosynthetic reactor; and (4) adjusting the pH value and temperature in the reactor, and collecting gas.

### TECHNICAL FIELD

The invention belongs to the technical field of environmental protection, and in particular relates to a method for adding nano titania to improve generation of hydrogen by photosynthetic bacteria with organic wastewater.

### BACKGROUND

Given that shortage of traditional mineral fuel energies is a growing problem, hydrogen has increasingly drawn extensive attention as a clean, pollution-free, green and new energy. Hydrogen results in no pollutant after combustion, and is high in quantity of combustion (142.35 KJ/g), which is 2.75 times as much as gasoline, 3.9 times as much as alcohol and 4.5 times as much as coke. In addition, hydrogen is also an important chemical raw material in many industrial productions. At present, physicochemical process and biological process are main acquisition ways for hydrogen. The physicochemical process itself requires consumption of a large amount of energy substances and also leads to relatively high cost. The biological process does no require consumption of a large amount of energy in hydrogen generation and has the advantages of low cost and good environment-friendly property. Top priority in the researches on the biological process for hydrogen generation is given to photosynthetic biological process for hydrogen generation and anaerobic fermentation process for hydrogen generation (e.g. International Journal of Hydrogen Energy, 2002, 27, 1315-1329; Enzyme and Microbial Technology, 2006, 38, 569-582). Hydrogen generation is realized by fermenting organic substances in wastewater or solid wastes, thus hydrogen is acquired while organic pollutants are degraded, furthermore, the hydrogen generating cost can be lowered and the problem of environmental pollution can be solved.

Photosynthetic bacteria are capable of photosynthesis and nitrogen fixation and have the hydrogen photoproduction capability under an anaerobic condition. There is only one photosynthesis center in the photosynthetic bacteria, thus bringing high photo-conversion efficiency and enormous hydrogen generating potential. The research has found that photosynthetic bacteria can convert a plurality of substrates, such as carbohydrates, low molecular weight organic acids and the like, into hydrogen under the action of light energy (e.g. International Journal of Hydrogen Energy, 2006, 31, 1585-1590). The current researches focus mainly on photosynthetic generation of hydrogen by low molecular weight organic acid and high-carbohydrate wastewater (e.g. International Journal of Hydrogen Energy, 2005, 30, 785-793; International Journal of Hydrogen Energy, 2006, 31, 1514-1521). These substrates are simple in composition and small in influence on photosynthetic bacteria. The conversion efficiency in generation of hydrogen by photosynthetic bacteria in these researches, though as high as 2 mol H₂/mol glucose, is still lower than a theoretical hydrogen generating amount, i.e. 12 mol H₂/mol glucose. The nano titania has photocatalysis features under the action of light energy and is able to decompose a macromolecular substance into micromolecular substances, but the documents focus mainly on treatment for organic substances that are seldom degradable (e.g. Journal of Hazardous Materials, 2009, 162, 1193-1198). The report that generation of hydrogen by photosynthetic bacteria with organic wastewater (e.g. brewing wastewater, sludge fermentation broth, food wastewater and the like) is facilitated by nano titania under the action of light energy has not been seen yet.

### SUMMARY OF THE INVENTION

An objective of the invention is to provide a method for facilitating generation of hydrogen by photosynthetic bacteria with organic wastewater, in which anaerobic hydrogen generation is achieved by photosynthetic bacteria under the action of light energy, and organic acids, proteins and carbohydrates in organic wastewater can be further converted into hydrogen by means of photocatalysis features of nano titania.

To reach the objective above, the technical solution in the invention is as follows: the hydrogen generating amount by photosynthetic bacteria with organic wastewater is increased by means of photocatalysis features of nano titania. The nano titania at a certain concentration is added to improve the bioactivity of photosynthetic bacteria and the activity of nitrogenase and reduce the activity of hydrogenase, in this way, the hydrogen generating ability of the photosynthetic bacteria is improved. Meanwhile, the nano titania can decompose macromolecular protein into micromolecular substances, which is conductive to use, growth and hydrogen generation of the photosynthetic bacteria.

The invention provides a method for increasing the hydrogen generating amount by photosynthetic bacteria with organic wastewater by means of photocatalysis features of nano titania, and the organic wastewater is wastewater containing rich glucose, starch, protein and volatile organic acids and the like, such as sludge fermentation broth, food wastewater, brewing wastewater and the like.
The method comprises the following specific steps:
(1) adding organic wastewater to a photosynthetic reactor.
(2) inoculating hydrogen generating photosynthetic bacteria to the photosynthetic reactor, wherein the inoculation concentration is 400-1000 mg/L.
(3) adding nano titania to the photosynthetic reactor, wherein the concentration is 50-150 mg/L; and
(4) adjusting the pH value to be within a range from 6 to 8 and the temperature to be within a range from 30° C. to 35° C. in the reactor, and collecting gas at the same time.

Wherein, the photosynthetic bacteria in the step (2) are photosynthetic bacteria capable of hydrogen generation (including, but not limited to, Rhodobacter sphaeroides, Rhodopseudomonas palustris, Rhodospirillum rubrum, Rhodobacter sphaeroides and Rhodomicrobium vannielii).

Wherein, the preferable condition for the invention is that: nano titania is added to increase the hydrogen generating amount by the photosynthetic bacteria.

With the technical solution above, the invention has the positive effects that:

(1) excellent environmental benefit is obtained; and the capability of hydrogen generation by biological photosynthesis is enhanced, which contributes to acquisition of more clean energy hydrogen;

(2) excellent economical benefit is obtained; on one hand, the hydrogen generating amount can be increased by adding nano titania; and on the other hand, hydrogen generation can be achieved by fermenting organic substances in the organic wastewater, therefore, the cost is saved due to concurrent progression of clean energy generation and organic wastewater treatment.

### DETAILED DESCRIPTION OF THE EMBODIMENTS

Further description is made below to the invention with reference to the embodiments.

### Embodiment 1

Supernatant is discharged after residual sludge from an urban sewage treatment plant is precipitated for 24 hours, and the sludge characteristics subsequent to concentration are as follows: total suspended solid (TSS) 17.34±1.58 g/L, volatile suspended solid (VSS) 12.50±1.09 g/L, soluble COD (SCOD) 145±23 mg/L, total COD (TCOD) 12400±260 mg/L, total organic carbon 1632±79 mg-COD/L, and total protein 6133±169 mg-COD/L. The sludge is put in a fermentation tank for alkaline fermentation for 8 days, and the fermentation broth is collected. The fermentation broth characteristics are as follows: COD 6856±342 mg-COD/L, total carbohydrate 770±38 mg-COD/L, total protein 2123±106mg-COD/L, VFA 2931±146 mg-COD/L, NH₄⁺-N 194±9 mg/L, and PO₄³⁻-p 108±5 mg/L.

The hydrogen generating capability of the photosynthetic bacteria is limited by high-concentration ammonia-nitrogen in the fermentation broth, so it must be removed at first. The ammonia-nitrogen in the sludge fermentation broth is recovered by a struvite recovery process, and the fermentation broth characteristics subsequent to recovery are as follows: COD 6300±315 mg-COD/L, total carbohydrate 730±36 mg-COD/L, total protein 1974±99 mg-COD/L, VFA 2638±131 mg-COD/L, NH₄⁺-N 27±1 mg/L, and PO₄³⁻-P 14+1 mg/L.

The specific steps of photosynthetic hydrogen generation are as follows:

The ammonia-nitrogen-recovered sludge fermentation broth above is added to glass-made photosynthetic reactors with a working volume of 0.5 L. Photosynthetic bacteria are inoculated to the reactor A at the concentration of 1000 mg/L. The reaction temperature is 30±1° C., the pH is adjusted to be within a range from 6 to 8, the illumination intensity is 50001ux, stirring is performed under an anaerobic condition, and the resultant gas is collected by a gas collecting bag. The composition contents of the gas are determined by gas chromatography GC112. Nano titania (at the concentration of 100 mg/L) and photosynthetic bacteria (at the concentration of 1000 mg/L) are added to the reactor B, and the rest operations are the same as those in the reactor A. Only nano titania is added to the reactor C with no inoculation of photosynthetic bacteria, and the rest operations are the same as those in the reactor A.

The total hydrogen generating amount in the reactor A is 78 ml, and the total hydrogen generating amount in the reactor B is 112 ml, which is increased by 43.6% compared with the hydrogen generating amount in the reactor A.

No hydrogen is generated in the reactor C and there is no phenomenon of photolytic hydrogen generation.

The final concentration of the photosynthetic bacteria in the reactor A is 1262 mg/L, and the final concentration of the photosynthetic bacteria in the reactor B is 1442 mg/L, which is increased by 14.3% compared with the final concentration of the photosynthetic bacteria in the reactor A.

The nitrogenase activity of the photosynthetic bacteria in the reactor A is 250 nmol C₂H₄/mg dry cell/h, and the nitrogenase activity in the reactor B is 320 nmol C₂H₄/mg dry cell/h, which is increased by 14.2% compared with the nitrogenase activity in the reactor A.

The hydrogenase activity of the photosynthetic bacteria in the reactor A is 518 U/mg dry cell, and the hydrogenase activity in the reactor B is 357 U/mg dry cell, which is decreased by 31.1% compared with the hydrogenase activity in the reactor A.

### Embodiment 2

The method for generating the sludge fermentation broth is the same as that in the Embodiment 1, and the fermentation broth characteristics are as follows: COD 5988±300 mg-COD/L, total carbohydrate 758±38 mg-COD/L, total protein 2214±106 mg-COD/L, VFA 2381±119 mg-COD/L, NH₄⁺-N 164±9 mg/L, and PO₄³⁻-P 92±5 mg/L.

The ammonia-nitrogen in the sludge fermentation broth is recovered by a struvite recovery process, and the fermentation broth characteristics subsequent to recovery are as follows: COD 5412±270 mg-COD/L, total carbohydrate 601±29 mg-COD/L, total protein 1747±87 mg-COD/L, VFA 2296±114 mg-COD/L, NH₄⁺-N 15±1 mg/L, and PO₄³⁻-P 12±1 mg/L.

The specific steps of photosynthetic hydrogen generation are as follows:

Organic wastewater is added to glass-made photosynthetic reactors with a working volume of 0.5 L. Photosynthetic bacteria are inoculated to the reactor A at the concentration of 400 mg/L. The operating conditions for the reactors and the gas collecting and measuring methods are as described in the Embodiment 1. Nano titania (at the concentration of 100 mg/L) and photosynthetic bacteria (at the concentration of 400 mg/L) are added to the reactor B, and the rest operations are the same as those in the reactor A in the Embodiment 2.

The total hydrogen generating amount in the reactor A is 59.8 ml, and the total hydrogen generating amount in the reactor B is 73.6 ml, which is increased by 23.1% compared with the hydrogen generating amount in the reactor A.

### Embodiment 3

Organic wastewater from a brewery has the characteristics as follows: COD 1800±90 mg-COD/L, acetic acid 1300±60 mg-COD/L, propionic acid 300±15 mg-COD/L, butyric acid 67±3 mg-COD/L and NH₄⁺-N 15±1 mg/L.

The specific steps of photosynthetic hydrogen generation are as follows:

Organic wastewater is added to glass-made photosynthetic reactors with a working volume of 0.5 L. Photosynthetic bacteria are inoculated to the reactor A at the concentration of 800 mg/L. The operating conditions for the reactors and the gas collecting and measuring methods are as described in the Embodiment 1. Nano titania (at the concentration of 150 mg/L) and photosynthetic bacteria (at the concentration of 800 mg/L) are added to the reactor B, and the rest operations are the same as those in the reactor A in the Embodiment 3.

The total hydrogen generating amount in the reactor A is 179 ml, and the total hydrogen generating amount in the reactor B is 214 ml, which is increased by 19.6% compared with the hydrogen generating amount in the reactor A.

### Embodiment 4

Organic wastewater from a bean products factory has the characteristics as follows: COD 25000 mg/L, starch 6000 mg/L, sucrose 700 mg/L, protein 500 mg/L, volatile acid 200 mg/L and NH₄⁺-N 29.5 mg/L.

The specific steps of photosynthetic hydrogen generation are as follows:

Organic wastewater is added to glass-made photosynthetic reactors with a working volume of 0.5 L. Photosynthetic bacteria are inoculated to the reactor A at the concentration of 1000 mg/L. The operating conditions for the reactors and the gas collecting and measuring methods are as described in the Embodiment 1. Nano titania (at the concentration of 50 mg/L) and photosynthetic bacteria (at the concentration of 1000 mg/L) are added to the reactor B, and the rest operations are the same as those in the reactor A in the Embodiment 4.

The total hydrogen generating amount in the reactor A is 376 ml, and the total hydrogen generating amount in the reactor B is 469 ml, which is increased by 24.8% compared with the hydrogen generating amount in the reactor A.

### Embodiment 5

Organic wastewater from a candy factory has the characteristics as follows: COD 6500 mg/L and BOD 4400 mg/L, and mainly contains glucose, sucrose and starch.

The specific steps of photosynthetic hydrogen generation are as follows:

Organic wastewater is added to glass-made photosynthetic reactors with a working volume of 0.5 L. Photosynthetic bacteria are inoculated to the reactor A at the concentration of 1000 mg/L. The operating conditions for the reactors and the gas collecting and measuring methods are as described in the Embodiment 1. Nano titania (at the concentration of 100 mg/L) and photosynthetic bacteria (at the concentration of 1000 mg/L) are added to the reactor B, and the rest operations are the same as those in the reactor A in the Embodiment 5.

The total hydrogen generating amount in the reactor A is 94 ml, and the total hydrogen generating amount in the reactor B is 126 ml, which is increased by 34.2% compared with the hydrogen generating amount in the reactor A.

The above description made to the embodiments is given for better understanding and application of the invention by those ordinary skilled in this art. Apparently, by those skilled who are familiar with this art, various modifications could be readily made to these embodiments and the general principal described herein could be applied to other embodiments without creative effort. Thus, the invention is not limited to the embodiments described herein, and improvements and modifications made without departing from the scope of the invention by those skilled in this art in accordance with the disclosure of the invention shall fall within the scope of the invention.

1. A method for facilitating generation of hydrogen by photosynthetic bacteria with organic wastewater, characterized in that: the hydrogen generating amount by photosynthetic bacteria with organic wastewater is increased by means of photocatalysis features of nano titania.

2. The method for facilitating generation of hydrogen by photosynthetic bacteria with organic wastewater according to claim 1, characterized in that: the method comprises the following specific steps:
(1) adding organic wastewater to a photosynthetic reactor;
(2) inoculating hydrogen generating photosynthetic bacteria to the photosynthetic reactor;
(3) adding nano titania to the photosynthetic reactor; and
(4) adjusting the pH value and temperature in the reactor, and collecting gas.

3. The method for facilitating generation of hydrogen by photosynthetic bacteria with organic wastewater according to claim 2, characterized in that: the concentration after inoculation of the photosynthetic bacteria in the step (2) is 400-1000 mg/L.

4. The method for facilitating generation of hydrogen by photosynthetic bacteria with organic wastewater according to claim 2, characterized in that: the concentration after addition of the nano titania in the step (3) is 50-150 mg/L.

5. The method for facilitating generation of hydrogen by photosynthetic bacteria with organic wastewater according to claim 2, characterized in that: in the step (4), the pH value is adjusted to be within a range from 6 to 8, and the temperature is adjusted to be within a range from 30° C. to 35° C.

6. The method for facilitating generation of hydrogen by photosynthetic bacteria with organic wastewater according to claim 2, characterized in that: the photosynthetic bacteria in the step (2) are photosynthetic bacteria capable of hydrogen generation.

7. The method for facilitating generation of hydrogen by photosynthetic bacteria with organic wastewater according to claim 6, characterized in that: the photosynthetic bacteria include:
Rhodobacter sphaeroides, Rhodopseudomonas palustris, Rhodospirillum rubrum, Rhodobacter sphaeroides and Rhodomicrobium vannielii.

8. The method for facilitating generation of hydrogen by photosynthetic bacteria with organic wastewater according to claim 2, characterized in that: the organic wastewater in the step (1) is wastewater containing rich glucose, starch, protein or volatile organic acid.
